(19) United States
(12) Patent Application Publication
(10) Pub. No.: US 2023/0190138 A1
(43) Pub. Date: Jun. 22, 2023

(54) INFORMATION PROCESSING APPARATUS, INFORMATION PROCESSING METHOD, PROGRAM, INFORMATION PROCESSING SYSTEMS, AND GENERATION METHOD

(71) Applicant: NEC Corporation, Tokyo (JP)

(72) Inventors: Chenhui Huang, Tokyo (JP); Kenichiro Fukushi, Tokyo (JP); Yusuke Sekiguchi, Miyagi (JP); Haruki Yaguchi, Miyagi (JP); Keita Honda, Miyagi (JP); Dai Owaki, Miyagi (JP); Shinichi Izumi, Miyagi (JP)

(73) Assignee: NEC Corporation, Tokyo (JP)

(21) Appl. No.: 18/081,893

(22) Filed: Dec. 15, 2022

(30) Foreign Application Priority Data
Dec. 21, 2021 (JP) 2021-207174

(51) Int. Cl. A61B 5/11; A61B 5/00

(52) U.S. Cl. CPC A61B 5/112; A61B 5/6807; A61B 2562/0219

(57) ABSTRACT

An information processing apparatus includes an acquisition unit configured to acquire information based on a sensor attached to a foot of a user, a determination unit configured to make a decision about an abnormality in walking of the user based on an acceleration of the user in a walking direction, acquired by the acquisition unit, and an output unit configured to output information based on a result of the decision made by the determination unit.

### INCORPORATION BY REFERENCE

This application is based upon and claims the benefit of priority from Japanese patent application No. 2021-207174, filed on Dec. 21, 2021, the disclosure of which is incorporated herein in its entirety by reference.

### TECHNICAL FIELD

The present disclosure relates to an information processing apparatus, an information processing method, a program, an information processing system, and a generation method.

### BACKGROUND ART

A technology for diagnosing (determining) an abnormality in a lower limb based on data measured by a sensor(s) has been known (see, for example, Japanese Unexamined Patent Application Publications No. 2004-261525 and No. 2019-150229).

### SUMMARY

However, the technology disclosed in Japanese Unexamined Patent Application Publications No. 2004-261525 and No. 2019-150229 has a problem that, in some cases, it cannot be properly determined that, for example, there is an abnormality in a lower limb.

In view of the above-described problem, an example object of the present disclosure is to provide an information processing apparatus, an information processing method, a program, and an information processing system capable of making a proper decision about an abnormality in a lower limb.

In a first example aspect, an information processing apparatus includes: an acquisition unit configured to acquire information based on a sensor attached to a foot of a user; a determination unit configured to make a decision about an abnormality in walking of the user based on an acceleration of the user in a walking direction, acquired by the acquisition unit; and an output unit configured to output information based on a result of the decision made by the determination unit.

Further, in a second example aspect, an information processing method includes: acquiring information based on a sensor attached to a foot of a user; making a decision about an abnormality in walking of the user based on an acquired acceleration of the user in a walking direction; and outputting information based on a result of the decision.

Further, in a third example aspect, a program causes an information processing apparatus to perform: a process for acquiring information based on a sensor attached to a foot of a user; a process for making a decision about an abnormality in walking of the user based on an acquired acceleration of the user in a walking direction; and a process for outputting information based on a result of the decision.

Further, in a fourth example aspect, an information processing system includes a sensor attached to a foot of a user, an information processing apparatus, and an information processing terminal. This information processing system includes: an acquisition unit configured to acquire information based on an acceleration of the user in a walking direction, measured by the sensor; a determination unit configured to make a decision about an abnormality in walking of the user based on an acceleration of the user in a walking direction, acquired by the acquisition unit; and an output unit configured to make the information processing terminal output information based on a result of the decision made by the determination unit.

Further, in a fifth example aspect, a generation method includes: acquiring a data set containing a combination of information based on an acceleration of a user in a walking direction, measured by a sensor attached to a foot of the user, and information indicating an abnormality in walking of the user; generating, based on an acquired acceleration of the user in the walking direction, a trained model from information measured by the sensor attached to the foot of the user, the trained model being configured to make a decision about an abnormality in walking of the user; and outputting the generated trained model.

### BRIEF DESCRIPTION OF DRAWINGS

The above and other aspects, features, and advantages of the present disclosure will become more apparent from the following description of certain example embodiments when taken in conjunction with the accompanying drawings, in which:

FIG. 1 shows an example of a configuration of an information processing apparatus that performs a generation process according to an example embodiment;

FIG. 2 shows an example of a configuration of an information processing apparatus that performs a determination process according to an example embodiment;

FIG. 3 shows an example of a configuration of an information processing system according to an example embodiment;

FIG. 4 shows an example of a hardware configuration of an information processing apparatus according to an example embodiment;

FIG. 5 shows an example of a position where a sensor according to an example embodiment is attached;

FIG. 6 shows an example of a configuration of a measurement apparatus according to an example embodiment;

FIG. 7 is a flowchart showing an example of a process for generating a trained model according to an example embodiment;

FIG. 8 shows an example of a data set for machine learning according to an example embodiment;

FIG. 9 shows an example of changes in the acceleration in the walking direction during a period in which a healthy user makes one step;

FIG. 10 shows an example of a walking motion of a healthy person;

FIG. 11 shows an example of a walking motion of a healthy person;

FIG. 12 shows an example of a walking motion of a healthy person;

FIG. 13 is a flowchart showing an example of a determination process performed by an information processing system according to an example embodiment;

FIG. 14 shows an example of changes in the acceleration in the walking direction during a period in which a healthy person makes one step and those during a period in which a patient makes one step based on data measured by a sensor according to an example embodiment;

FIG. 15 shows an example of frequency components of an acceleration in the walking direction during a period in which a healthy person makes one step and those during a period in which a patient makes one step based on data measured by a sensor according to an example embodiment;

FIG. 16 shows an example of changes in an angle between the sole of a foot and the ground surface in the walking direction during a period in which a healthy person makes one step and those during a period in which a patient makes one step based on data measured by a sensor according to an example embodiment;

FIG. 17 shows an example of frequency components of an acceleration in the vertically upward direction during a period in which a healthy person makes one step and those during a period in which a patient makes one step based on data measured by a sensor according to an example embodiment;

FIG. 18 shows an example of the value of a ratio between accelerations A and B during a period in which a healthy person makes one step and that during a period in which a patient makes one step based on data measured by a sensor according to an example embodiment;

FIG. 19 shows an example of the value of a ratio between accelerations A and C during a period in which a healthy person makes one step and that during a period in which a patient makes one step based on data measured by a sensor according to an example embodiment;

FIG. 20 shows an example of a one-vibration component of an acceleration in the walking direction during a period in which a healthy person makes one step and that during a period in which a patient makes one step based on data measured by a sensor according to an example embodiment;

FIG. 21 shows an example of a difference between the maximum value and the minimum value of an angle between the sole of a foot and the ground surface in the walking direction during a period in which a healthy person makes one step and that during a period in which a patient makes one step based on data measured by a sensor according to an example embodiment; and

FIG. 22 shows an example of a five-vibration component of an acceleration in the vertically upward direction during a period in which a healthy person makes one step and that during a period in which a patient makes one step based on data measured by a sensor according to an example embodiment.

### EXAMPLE EMBODIMENT

A principle of the present disclosure will be described with reference to several illustrative example embodiments. It should be understood that these example embodiments are described only for an illustrative purpose and will assist those skilled in the art in understanding and carrying out the present disclosure without suggesting any limitations in regard to the scope of the disclosure. Disclosures described in this specification can also be implemented in a variety of ways other than those described below.

In the following description and the claims, unless otherwise defined, all technical and scientific terms used in this specification have the same meanings as those generally understood by those skilled in the technical field to which the present disclosure belongs.

An example embodiment according to the present disclosure will be described hereinafter with reference to the drawings.

### (First Example Embodiment)

### <Configuration>

«Information Processing Apparatus 10A that Performs Generation Process»

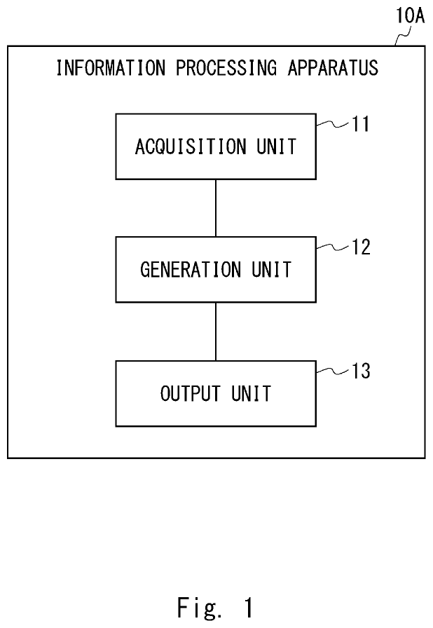

A configuration of an information processing apparatus 10A that performs a generation process according to an example embodiment will be described with reference to FIG. 1. FIG. 1 shows an example of the configuration of the information processing apparatus 10A that performs a generation process according to the example embodiment. The information processing apparatus 10A (e.g., a server 40 described later) includes an acquisition unit 11, a generation unit 12, and an output unit 13. Each of these components may be implemented through cooperation between at least one program installed in the information processing apparatus 10A and hardware such as a processor 101, a memory 102, and the like of the information processing apparatus 10A.

The acquisition unit 11 acquires various types of information from a storage unit disposed inside the information processing apparatus 10A or from an external apparatus. The acquisition unit 11 acquires, for example, a data set containing a combination of information based on an acceleration in the walking direction (the traveling direction) of a user, measured by a sensor(s) attached to a foot (or feet) of the user, and information indicating an abnormality in the walking of the user.

Based on the information acquired by the acquisition unit 11, the generation unit 12 generates a trained model that makes a decision about an abnormality in the walking of the user from the information measured (i.e., obtained) by the sensor attached to the foot of the user. The output unit 13 outputs (transmits or records) various types of information to the storage unit disposed inside the information processing apparatus 10A or to the external apparatus. For example, the output unit 13 outputs the trained model generated by the generation unit 12.

«Information Processing Apparatus 10B that Performs Determination Process»

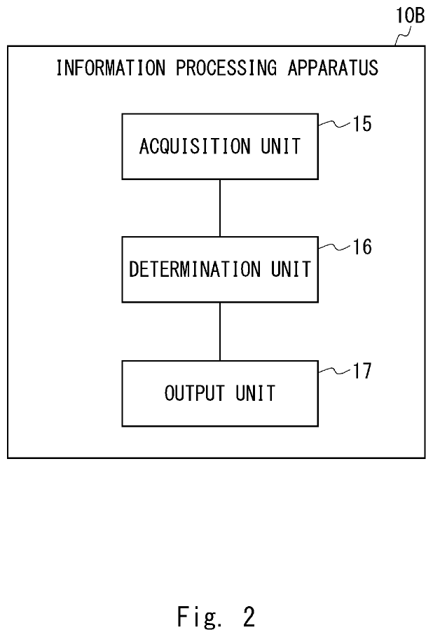

Next, a configuration of an information processing apparatus 10B according to an example embodiment will be described with reference to FIG. 2. FIG. 2 shows an example of the configuration of the information processing apparatus 10B that performs a determination process according to the example embodiment. The information processing apparatus 10B (e.g., a server 40 described later) includes an acquisition unit 15, a determination unit 16, and an output unit 17. Each of these components may be implemented through cooperation between at least one program installed in the information processing apparatus 10B and hardware such as a processor 101, a memory 102, and the like of the information processing apparatus 10B. Note that the same information processing apparatus (i.e., one information processing apparatus) may serve as both the information processing apparatuses 10A and 10B, or they may be different information processing apparatuses (i.e., separate information processing apparatuses). Note that, in the following description, when it is unnecessary to distinguish between the information processing apparatuses 10A and 10B, each of them may also be referred to simply as the “information processing apparatus 10”.

The acquisition unit 15 acquires various types of information from a storage unit disposed inside the information processing apparatus 10B or from an external apparatus. The acquisition unit 15 acquires, for example, information based on an acceleration of a user in the walking direction, measured by a sensor(s) attached to a foot (or feet) of the user.

The determination unit 16 makes a decision about an abnormality in the walking of the user based on the information acquired by the acquisition unit 15. The output unit 17 outputs (transmits, displays, reports, or records) various types of information to the storage unit disposed inside the information processing apparatus 10B or to the external apparatus. For example, the output unit 17 outputs information based on the result of the decision made by the determination unit 16.

### (Second Example Embodiment)

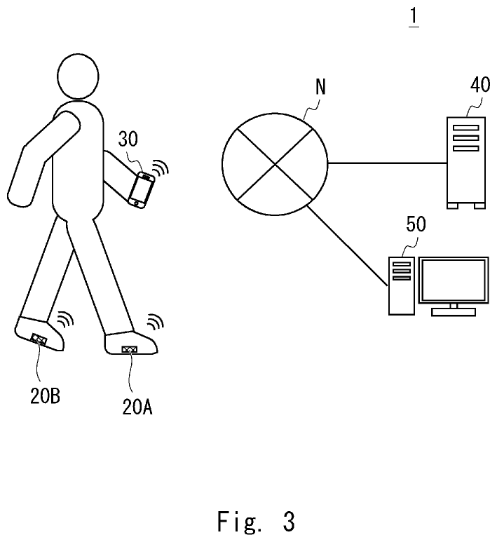

Next, a configuration of an information processing system 1 according to an example embodiment will be described with reference to FIG. 3.

### <System Configuration>

FIG. 3 shows an example of a configuration of an information processing system 1 according to an example embodiment. In the example shown in FIG. 3, the information processing system 1 includes a measurement apparatus 20A, and a measurement apparatus 20B (hereinafter, when it is unnecessary to distinguish between them, each of them may also be referred to simply as the “measurement apparatus 20”). Further, the information processing system 1 includes a user terminal 30, a server 40, and a hospital terminal 50. Note that the numbers of the measurement apparatuses 20, the user terminal 30, the server 40, and the hospital terminal 50 is not limited to the numbers shown in the example shown in FIG. 3. Note that each of the measurement apparatuses 20, the user terminal 30, the server 40, and the hospital terminal 50 is an example of the information processing apparatus 10.

An example in which a process for generating (training) a trained model, and a determination (estimation or inference) process are performed in the server 40 will be described hereinafter. Note that the generation process may be performed in at least one of the measurement apparatuses 20, the user terminal 30, the server 40, and the hospital terminal 50. Further, the determination process may be performed in at least one of the measurement apparatuses 20, the user terminal 30, the server 40, and the hospital terminal 50. The generation process and the determination process may be performed in the same apparatus or in different apparatuses. The measurement apparatuses 20 and the user terminal 30 may be connected to each other through, for example, short-range wireless communication such as BLE (Bluetooth (Registered Trademark) Low Energy) or through a cable so that they can communicate with each other.

In the example shown in FIG. 3, the user terminal 30, the server 40, and the hospital terminal 50 are connected to each other through a network N so that they can communicate with each other. Examples of the network N include the Internet, a mobile communication system, a wireless LAN (Local Area Network), short-range wireless communication such as BLE, a LAN, and a bus. Examples of the mobile communication system include a fifth-generation mobile communication system (5G), a fourth-generation mobile communication system (4G), and a third-generation mobile communication system (3G).

The measurement apparatus 20 (i.e., each of the measurement apparatuses 20) includes a sensor 21 that is attached to a foot of a user. The measurement apparatus 20 outputs data measured (i.e., obtained) by using the sensor 21 to an external apparatus such as the user terminal 30.

The user terminal 30 may be, for example, an apparatus such as a smartphone, a tablet-type computer, a personal computer, an Internet of Things (IoT) communication device, and a mobile phone. The user terminal 30 transmits the data acquired from the measurement apparatus 20 to the server 40.

The server 40 is, for example, an apparatus such as a server, a cloud computing system, a personal computer, and a smartphone. The server 40 makes a decision about an abnormality in the walking of the user based on the data measured by the measurement apparatus 20. Further, the server 40 transmits information based on the result of the decision to the hospital terminal 50.

The hospital terminal 50 may be, for example, an apparatus such as a smartphone, a tablet-type computer, a personal computer, and a mobile phone. The hospital terminal 50 may be a terminal used in a facility such as a hospital. The hospital terminal 50 displays information received from the server 40 on a screen.

### <Hardware Configuration>

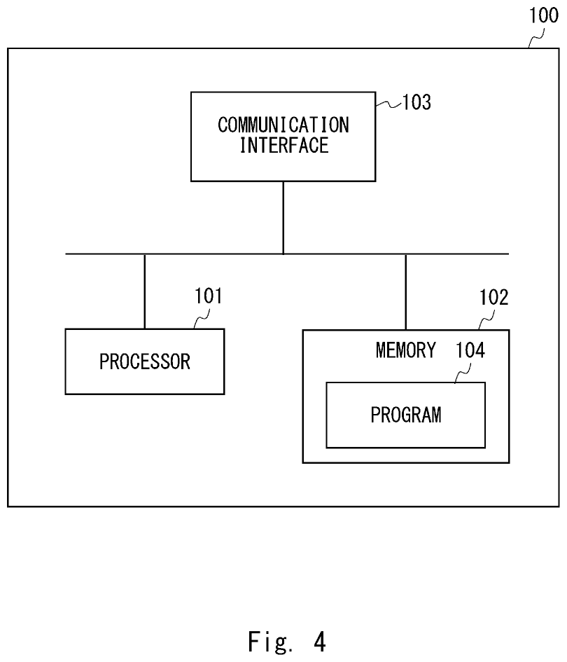

FIG. 4 shows an example of a hardware configuration of an information processing apparatus 10 according to an example embodiment. In the example shown in FIG. 4, the information processing apparatus 10 (a computer 100) includes a processor 101, a memory 102, and a communication interface 103.

These components may be connected to each other through a bus or the like. The memory 102 stores at least a part of a program 104. The communication interface 103 includes an interface necessary for communicating with other network elements.

When the program 104 is executed through the cooperation of the processor 101, the memory 102, and the like, at least one of the processes of the example embodiment according to the present disclosure is performed by the computer 100. The memory 102 may be any type of memory suitable for a local technology network. The memory 102 may be, but is not limited to, a non-transitory computer readable storage medium. Further, the memory 102 may be implemented by using any suitable data storage technology such as a semiconductor-based memory device, a magnetic memory device and a system, an optical memory device and a system, a fixed memory, and a removable memory. Although only one memory 102 is provided in the computer 100, a plurality of physically different memory modules may be provided in the computer 100. The processor 101 may be any type of processor. The processor 101 may include at least one of a general-purpose computer, a dedicated computer, a microprocessor, and a digital signal processor (DSP: Digital Signal Processor), and may also include, but is not limited to, at least one processor based on a multi-core processor architecture. The computer 100 may include a plurality of processors, such as an application-specific integrated circuit chip that is temporally dependent on a clock for synchronizing the main processor.

An example embodiment according to the present disclosure may be implemented by hardware, a dedicated circuit, software, a logic, or any combination thereof. In some aspects, an example embodiment may be implemented by hardware, while in other aspects, an example embodiment may be implemented by firmware or software that may be executed by a controller, a microprocessor, or other computing devices. The present disclosure also provides at least one computer program product that is tangibly stored in a non-transitory computer readable storage medium. The computer program product contains computer executable instructions, such as those contained in program modules, and is executed by a target real processor or by a device on a virtual processor, so that a process(es) or a method according to the present disclosure is performed. The program module contains routines, programs, libraries, objects, classes, components, and data structures for performing specific tasks or implementing specific abstract data types. The functions of the program module may be combined with those of the other program modules, or divided into a plurality of program modules as desired in various example embodiments. The machine executable instructions in the program module can be executed locally or in a distributed device(s). In the distributed device, the program module can be disposed on both local and remote storage media.

The program codes for performing the method according to the present disclosure may be written in any combination of at least one programming language. These program codes are provided to a processor or a controller of a general-purpose computer, a dedicated computer, or other programmable data processing apparatuses. When such a program code is executed by the processor or the controller, a function/operation in a flowchart and/or a block diagram to be implemented is executed. The program code is entirely executed in a machine, partially executed in a machine as a standalone software package, partially executed in a machine, partially executed in a remote machine, or entirely executed in a remote machine or a server.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g., magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

### <Measurement Apparatus 20>

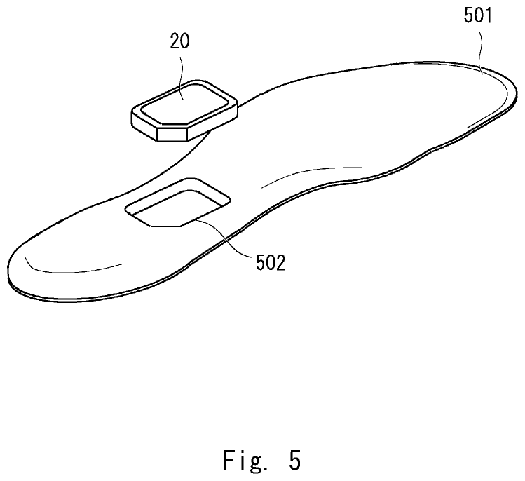

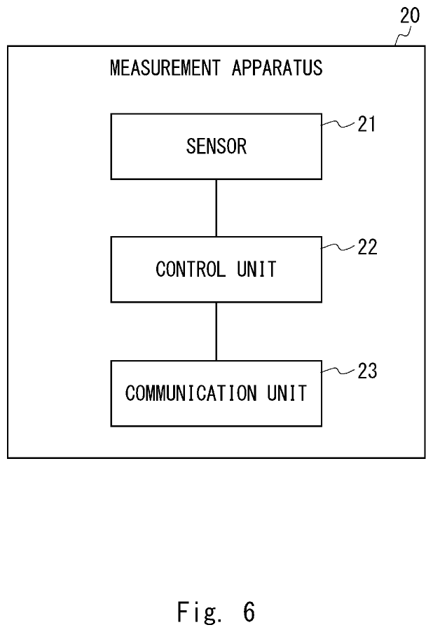

Next, an example of a measurement apparatus 20 according to an example embodiment will be described with reference to FIGS. 5 and 6. FIG. 5 shows an example of a position where a sensor 21 according to an example embodiment is attached. FIG. 6 shows an example of a configuration of the measurement apparatus 20 according to the example embodiment.

In the example shown in FIG. 5, the measurement apparatus 20 is housed (installed) in a recessed part 502 in an insole 501 of a shoe a user wears. Note that the sensor 21 of the measurement apparatus 20 may be attached to a position in the insole corresponding to a part of the underside of the user's foot between the arch of the foot and the heel thereof.

In the example shown in FIG. 6, the measurement apparatus 20 includes the sensor 21, a control unit 22, and a communication unit 23. The sensor 21 measures, for example, an acceleration and an angular velocity. The sensor 21 may be, for example, an inertial measurement unit (IMU: Inertial Measurement Unit) equipped with a three-direction acceleration sensor and a three-axis gyroscopic sensor. The control unit 22 outputs data measured (i.e., obtained) by using the sensor 21 to an external apparatus by using the communication unit 23. The control unit 22 may have a configuration similar to that of the computer 100 shown in FIG. 4. In this case, the control unit 22 may be, for example, a microcontroller.

### <Processing>

### «Generation Process»

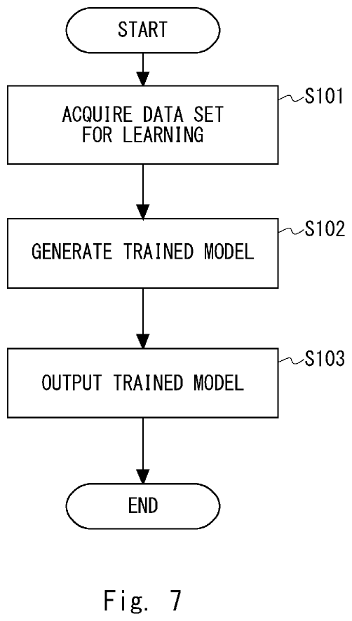

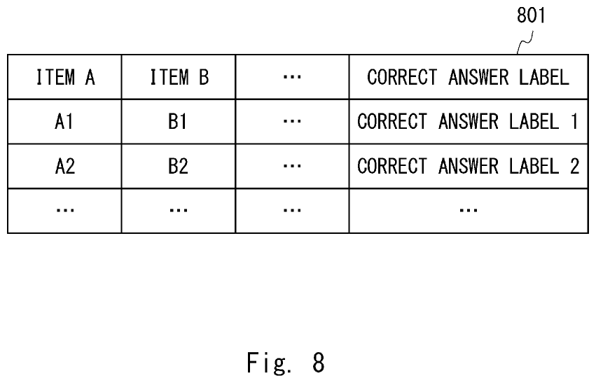

Next, with reference to FIGS. 7 and 8, an example of a process for generating a trained model (a learning process) performed by the information processing apparatus 10A according to the example embodiment will be described.

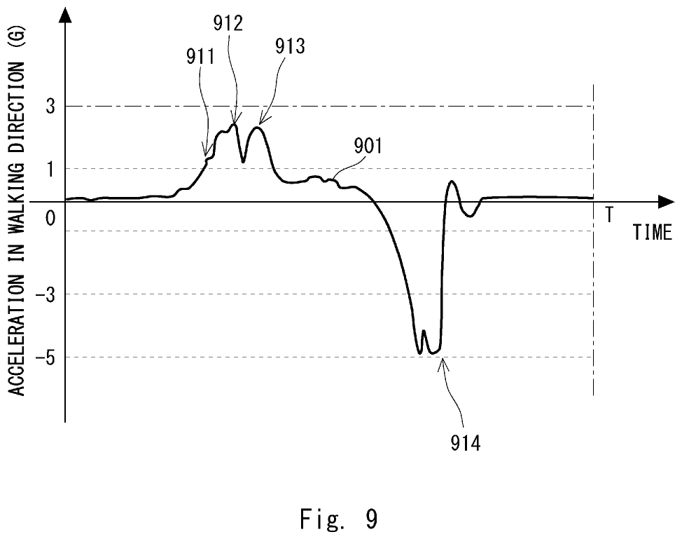

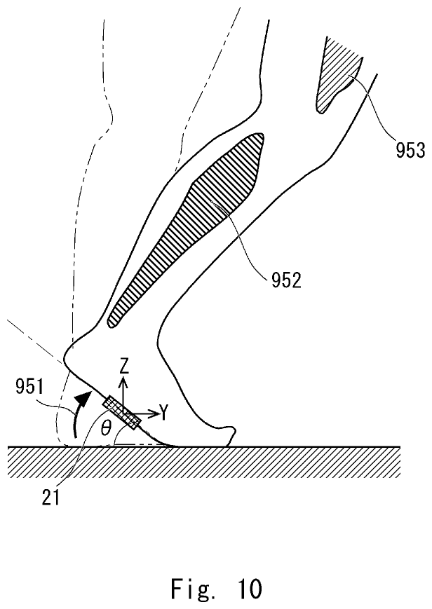

FIG. 7 is a flowchart showing an example of a process for generating a trained model according to an example embodiment. FIG. 8 shows an example of a data set for learning according to the example embodiment. FIG. 9 shows an example of changes in the acceleration in the walking direction during a period in which a healthy user makes one step. FIGS. 10 to 12 show an example of a walking motion of a healthy person.

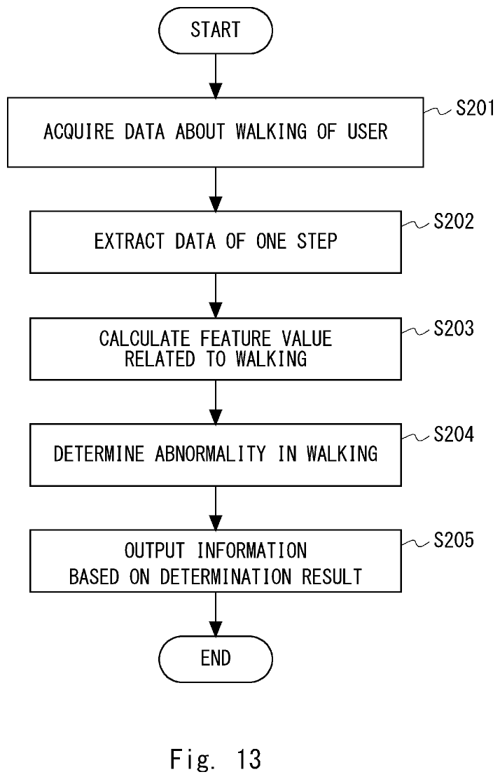

In a step S101, the acquisition unit 11 of the information processing apparatus 10A acquires a data set 801 for learning (hereinafter also referred to as the learning data set 801). In the example shown in FIG. 8, each record in the learning data set 801 contains data for a respective one of at least one item indicating a feature value related to walking, and its correct answer label. The data of each item may be data calculated based on data of at least one of an acceleration and an angular velocity measured by the sensor 21. The data of each item may be calculated, for example, any of the measurement apparatuses 20, the user terminal 30, the server 40, and the like through processes similar to those in steps S202 and S203 shown in FIG. 13 (which will be described later).

The correct answer label is a correct answer label used in supervised learning. The correct answer label may contain, for example, information indicating the presence/absence of an abnormality. Further, the correct answer label may include, for example, information indicating the severity of an abnormality. In this case, for example, the severity of an abnormality may be expressed by a number on a scale of 10 from 0 to 9, in which the severity “0” may indicate that there is no abnormality and the severity “9” may indicate the highest severity. The correct answer label may be, for example, designated (set or registered) by an operator of the server 40, a user of the hospital terminal 50 (e.g., a doctor or the like), or a user of the user terminal 30.

### (Walking Motion of Healthy Person)

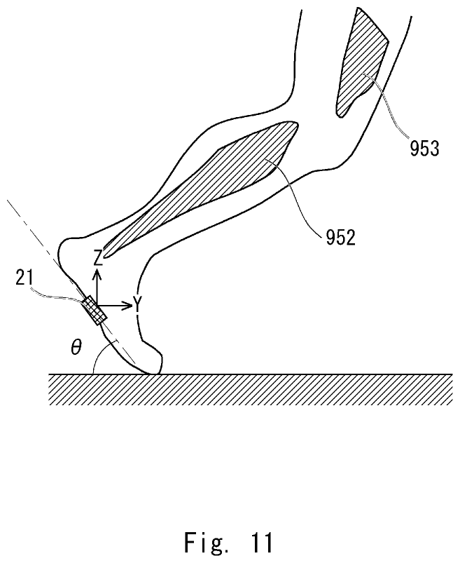

FIG. 9 shows an example of changes 901 in the acceleration in the walking direction during a period from a time 0 to a time T, i.e., during a period in which a healthy user makes one step. Note that, in FIGS. 10 to 12, the walking direction is parallel to the Y-direction, and the vertically upward direction is parallel to the-Z direction. Further, the angle between the sole of the foot and the ground surface in the walking direction is represented by θ. In the example shown in FIG. 9, at a time point 911, as shown in FIG. 10, a thigh muscle 953 is not worked (i.e., is in a relaxed state), while a lower-leg muscle 952 is worked (i.e., is in a strained state) and hence has contracted, so that the heel of the user is lifted from the ground surface as indicated by an arrow 951. Further, at a time point 912, as shown in FIG. 11, both the thigh muscle 953 and the lower-leg muscle 952 are not worked (i.e., are in relaxed states), so that the toe of the foot of the user comes off the ground surface (Toe off). Note that, in the following description, the time point at which the toe of the user's foot comes off the ground surface as shown in FIG. 11 is also referred to as a “time point B”. Further, the value of the acceleration in the walking direction at the time point B is also referred to as an “acceleration B” (an example of the “first acceleration”).

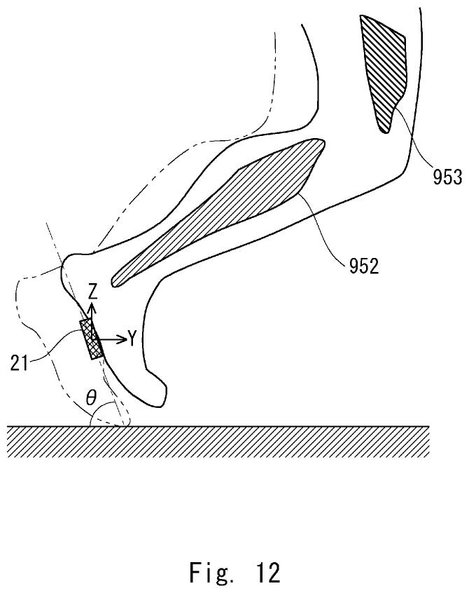

Further, at a time point 913, as shown in FIG. 12, the thigh muscle 953 is worked and hence has contracted, while the lower-leg muscle 952 is not worked, so that the entire lower limb of the user is swung forward. Note that, in the following description, the time point at which the entire lower limb of the user is swung forward as shown in FIG. 12 is also referred to as a “time point A”. Further, the value of the acceleration in the walking direction at the time point A is also referred to as an “acceleration A” (an example of the “second acceleration”). As shown in FIG. 9, in the case of the walking of a healthy person, changes 901 in the acceleration in the walking direction has a W-shaped waveform in which there are peaks at two time points, i.e., the time point 912 at which the toe of the user's foot comes off the ground surface and the time point 913 at which the entire lower limb of the user is swung forward. It is considered that this W-shaped waveform is obtained because sufficient Forefoot Rocker and sufficient knee flexion to smoothly accelerate the toe of the user's foot are performed at the time point 912, at which the toe comes off the ground surface.

Further, at a time point 914, the foot is sharply decelerated by decisively returning the knee from the flexed state to the stretched state before the foot lands on the ground surface. In the following description, the time point at which the foot of the user lands on the ground surface is also referred to as a “time point C”. Further, the value of the acceleration in the walking direction at the time point C is also referred to as an “acceleration C” (an example of the “third acceleration”). (Example in which acceleration B is used)

The items of the data included in the records of the learning data set 801 may include, for example, an item for data based on the acceleration B. This is because it is considered that since a user (a patient) suffering from osteoarthritis (OA) or the like may not be able to flex his/her knee sufficiently, an acceleration in the walking direction when the toe comes off the ground surface is degenerated (decreased) as compared to that of a healthy person.

In this case, the aforementioned item may be an item based on the acceleration B, and at least one of the acceleration A and the walking speed of the user. In this case, the item may include an item based on a ratio between the accelerations A and B (e.g., the value of the ratio between the accelerations A and B, or a reciprocal value thereof). This is because since there is no significant difference between the acceleration A of a patient suffering from OA or the like and that of a healthy person, the effect that would otherwise be caused by the difference between walking speeds is reduced for the value of the ratio between the accelerations A and B, or the reciprocal value thereof. Further, the item for data based on the acceleration B may include an item based on a ratio between the walking speed and the acceleration B (e.g., a radio expressed as “walking speed/acceleration B” or a reciprocal value thereof). Note that the walking speed may be calculated based on changes in the acceleration in the walking direction, measured by the sensor 21. Note that the symbol “/” indicates a division. Therefore, for example, an expression “X/Y” refers to the value of a ratio between X and Y.

(Example in which acceleration C is used)

The items of the data included in the records of the learning data set 801 may include, for example, an item for data based on the acceleration C. This is because it is considered that since a patient suffering from osteoarthritis (OA) or the like does not have an ability to move his/her knee sufficiently, he/she gradually decreases the walking speed during the swing phase, so that the acceleration C is degenerated (decreased) as compared to that of a healthy person. In this case, the aforementioned item may be an item based on the acceleration C, and at least one of the acceleration A and the walking speed of the user. In this case, the item may include an item based on a ratio between the accelerations A and C (e.g., a value expressed as “acceleration A/acceleration C”, or a reciprocal value thereof). This is because since there is no significant difference between the acceleration A of a patient suffering from OA or the like and that of a healthy person, the effect that would otherwise be caused by the difference between walking speeds is reduced for the value of the ratio between the accelerations A and C, or the reciprocal value thereof. Further, the item for data based on the acceleration C may include an item based on a ratio between the walking speed and the acceleration C (e.g., a radio expressed as “walking speed/acceleration C” or a reciprocal value thereof).

Further, the items of the data included in the records of the learning data set 801 may include, for example, an item for the magnitude of a one-vibration component of an acceleration of the user in the walking direction during a period in which the user makes one step. Further, the items of the data included in the records of the learning data set 801 may include, for example, an item based on the angle between the sole of the foot and the ground surface in the walking direction during a period in which the user makes one step. This is because it is considered that since a patient suffering from osteoarthritis (OA) or the like may not be able to flex (dorsiflex and plantar-flex) his/her ankle sufficiently, the angle between the sole of the foot and the ground surface in the walking direction is degenerated (decreased) as compared to that of a healthy person.

Further, the items of the data included in the records of the learning data set 801 may include, for example, the magnitude of a five-vibration component of an acceleration of the user in the vertical direction during a period in which the user makes one step.

Next, the generation unit 12 of the information processing apparatus 10A generates, based on the machine-learning data set 801, a trained model by using a machine learning method through supervised learning (Step S102). Note that the information processing apparatus 10A may use, for example, a weighted K-Nearest Neighbor Algorithm. In this case, the information processing apparatus 10A may generate, for example, a trained model in which weights that are inversely proportional to the Euclidean distance are added, and a K value is 10 (nearby 10 points).

Further, the information processing apparatus 10A may generate, for example, a trained model by using an arbitrary machine learning method through supervised learning, such as, a neural network (Neural Network: NN), a random forest, a linear regression, and a support vector machine.

Next, the output unit 13 of the information processing apparatus 10A outputs the trained model generated by the generation unit 12 (Step S103). Note that the information processing apparatus 10A may store the generated trained model in a storage unit provided therein. Alternatively or additionally, the information processing apparatus 10A may transmit (distribute) the generated trained model to an external apparatus(es) and make the external apparatus(es) record the trained model therein.

### «Determination Process»

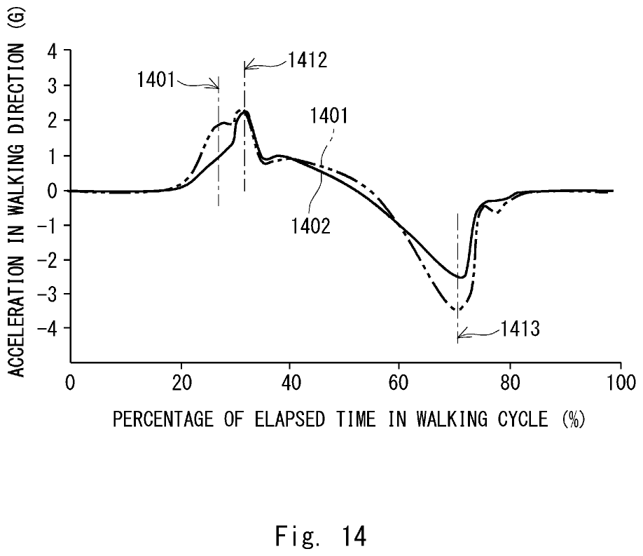

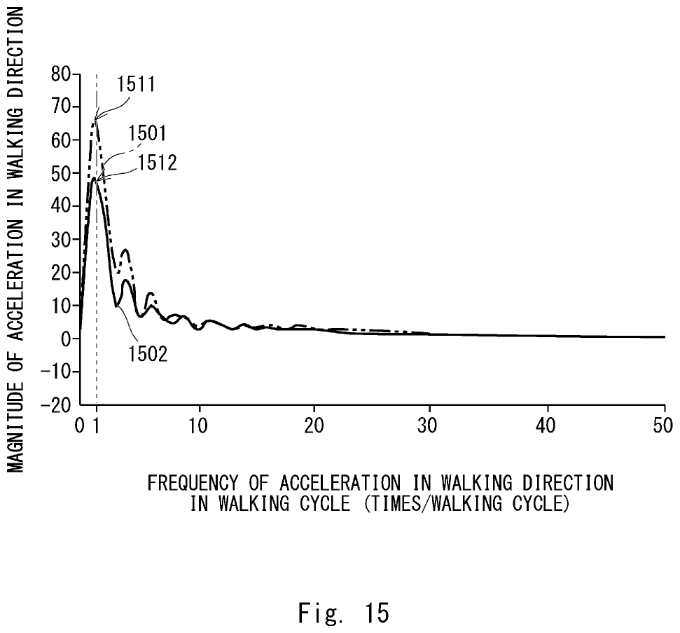

Next, an example of a determination process performed by an information processing apparatus 10B according to an example embodiment will be described with reference to FIG. 13. FIG. 13 is a flowchart showing an example of a determination process performed by an information processing system 1 according to an example embodiment. FIG. 14 shows an example of changes in the acceleration in the walking direction during a period in which a healthy person makes one step and those during a period in which a patient makes one step based on data measured (i.e., obtained) by the sensor 21 according to the example embodiment. FIG. 15 shows an example of frequency components of the acceleration in the walking direction during a period in which a healthy person makes one step and those during a period in which a patient makes one step based on data measured by the sensor 21 according to the example embodiment. FIG.

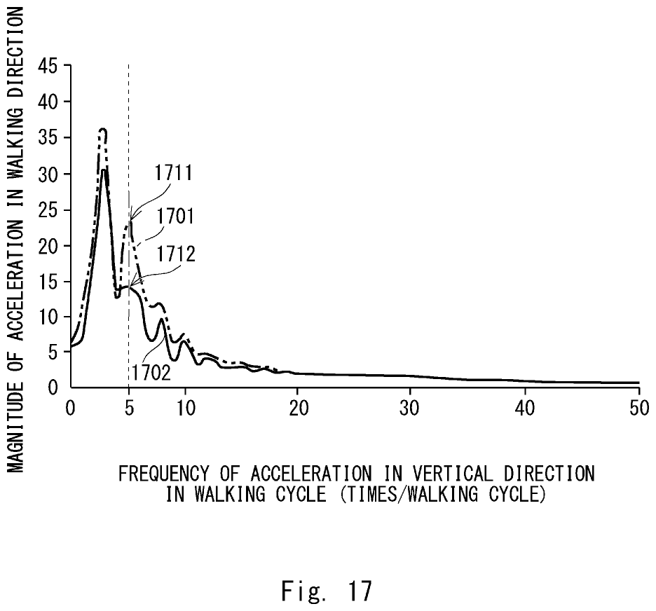

16 shows an example of changes in the angle between the sole of a foot and the ground surface in the walking direction during a period in which a healthy person makes one step and those during a period in which a patient makes one step based on data measured by the sensor 21 according to the example embodiment. FIG. 17 shows an example of frequency components of the acceleration in the vertically upward direction during a period in which a healthy person makes one step and those during a period in which a patient makes one step based on data measured by the sensor 21 according to the example embodiment.

In a step S201, the acquisition unit 15 of the information processing apparatus 10B acquires data about walking of a user, measured by the sensor 21. Next, the determination unit 16 of an information processing apparatus 10B extracts data about the walking of the user during a period in which the user makes one step (Step S202). Note that the information processing apparatus 10B may determine, for example, a start point and an end point of the period in which the user makes one step based on changes of the angle between the sole of the user's foot and the ground surface in the walking direction. In this case, the information processing apparatus 10B may determine the start point and the end point of the period in which the user makes one step based on, for example, a period from when the angle between the sole of the user's foot and the ground surface in the walking direction reaches the maximum value to when the angle, after reaching the minimum value, reaches the maximum value again.

Alternatively, the information processing apparatus 10B may determine the start point and the end point of the period in which the user makes one step based on, for example, changes in the acceleration in the walking direction. In this case, the information processing apparatus 10B may determine the start point and the end point of the period in which the user makes one step based on, for example, a period from when the acceleration in the walking direction reaches the maximum value to when the acceleration, after reaching the minimum value, reaches the maximum value again.

Then, the information processing apparatus 10B may normalize the length of the time period in which the user makes one step (i.e., the walking cycle) as indicated by the horizontal axis in FIG. 14. In this way, for example, it is possible to make a decision about an abnormality in the walking based on data that is measured with an arbitrary walking speed. Then, the information processing apparatus 10B extracts an acceleration A, an acceleration B, and an acceleration C from the time-length-normalized data during the period in which the user makes one step. FIG. 14 shows an example of changes 1401 in the acceleration in the walking direction during a period in which a healthy person makes one step, and changes 1402 in the acceleration in the walking direction during a period in which a patient suffering from OA or the like (hereinafter also referred to as an OA patient or the like) makes one step. In the example shown in FIG. 14, a time point 1411 corresponds to a time point B, and a time point 1412 corresponds to a time point A. Further, a time point 1413 corresponds to a time point C.

In the case where, after the length of the time period in which the user makes one step is normalized into a value irrelevant to the time, for example, there are two maximum values of the acceleration in the walking direction side by side in a predetermined range, the information processing apparatus 10B may determine the second maximum value as the time point A. In this way, for example, it is possible to appropriately determine the time point A for the healthy person. Alternatively, the information processing apparatus 10B may determine, for example, a time point at which the acceleration in the walking direction has the maximum value in the period in which the user makes one step as the time point A.

Further, in the case where, after the length of the time period in which the user makes one step is normalized into a value irrelevant to the time, for example, there are two maximum values of the acceleration in the walking direction side by side in a predetermined range, the information processing apparatus 10B may determine the first maximum value as the time point B. In this way, for example, it is possible to appropriately determine the time point B for the healthy person. Further, the information processing apparatus 10B may determine, for example, after the length of the time period in which the user makes one step is normalized into a value irrelevant to the time, a time point that is a predetermined length (i.e., a time equivalent to 5% of the one walking cycle) earlier than the time point at which the acceleration in the walking direction has the maximum value as the time point B.

Alternatively, the information processing apparatus 10B may determine, for example, a time point at which the acceleration in the walking direction has the minimum value in the period in which the user makes one step as the time point C.

Next, the determination unit 16 of the information processing apparatus 10B calculates feature values related to the walking based on the data about the walking of the user during the period in which the user makes one step (Step S203). Note that the information processing apparatus 10B calculates data for at least one item that is the same as an item(s) of the data included in the records of the learning data set 801 shown in FIG. 8. Therefore, the information processing apparatus 10B may calculate, for example, at least one of a ratio between the accelerations A and B and a ratio between the accelerations A and C.

Further, the information processing apparatus 10B may calculate, for example, the magnitude of a one-vibration component of an acceleration of the user in the walking direction during a period in which the user makes one step as shown in FIG. 15. In this case, the information processing apparatus 10B may first perform a Fourier transform for a waveform representing the changes in the acceleration of the user in the walking direction during the period in which the user makes one step, and thereby converts the changes in the acceleration from the time-dependent waveform into a frequency-dependent waveform. Then, the information processing apparatus 10B may extract the value of this waveform at a frequency at which one vibration occurs during the period in which the user makes one step (i.e., the frequency is one) as the one-vibration component. FIG. 15 shows an example of a frequency component 1501 of the acceleration in the walking direction during a period in which a healthy person makes one step, and a frequency component 1502 of the acceleration in the walking direction during a period in which an OA patient or the like makes one step. Further, FIG. 15 shows an example of a one-vibration component 1511 of the frequency component 1501 of a healthy person, and a one-vibration component 1512 of the frequency component 1502 of an OA patient or the like.

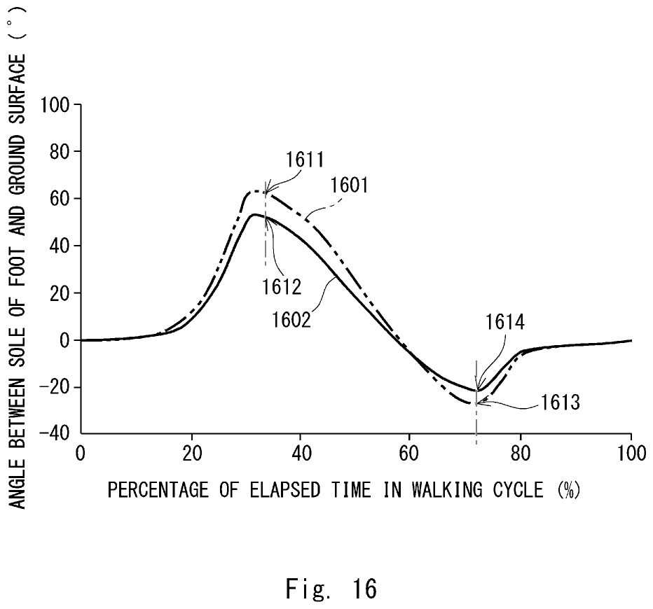

Further, the information processing apparatus 10B may calculate an item based on, for example, the angle between the sole of the foot and the ground surface in the walking direction during a period in which the user makes one step. In this case, this item may include, for example, a difference between the maximum value and the minimum value of the angle between the sole of the foot and the ground surface in the walking direction during the period in which the user makes one step. FIG. 16 shows an example of changes 1601 in the angle between the sole of the foot and the ground surface in the walking direction during a period in which a healthy person makes one step, and changes 1602 in the angle between the sole of the foot and the ground surface in the walking direction during a period in which an OA patient or the like makes one step. Further, FIG. 16 shows an example of a maximum value 1611 and a minimum value 1613 of the changes 1601 of the healthy person, and a maximum value 1612 and a minimum value 1614 of the changes 1602 of the OA patient or the like.

Further, the information processing apparatus 10B may calculate, for example, the magnitude of a five-vibration component of an acceleration of the user in the vertical direction during a period in which the user makes one step. In this case, the information processing apparatus 10B may first perform a Fourier transform for a waveform representing the changes in the acceleration of the user in the vertical direction during the period in which the user makes one step, and thereby converts the changes in the acceleration from the time-dependent waveform into a frequency-dependent waveform. Then, the information processing apparatus 10B may extract the value of this waveform at a frequency at which five vibrations occur during the period in which the user makes one step as the five-vibration component. FIG. 17 shows an example of a frequency component 1701 of the acceleration in the vertically upward direction during a period in which a healthy person makes one step, and a frequency component 1702 of the acceleration in the vertically upward direction during a period in which an OA patient or the like makes one step. Further, FIG. 17 shows an example of a five-vibration component 1711 of the frequency component 1701 of a healthy person, and a five-vibration component 1712 of the frequency component 1702 of an OA patient or the like.

Next, the determination unit 16 of the information processing apparatus 10B makes a decision (an estimation or inference) an abnormality in the walking of the user based on the data of the calculated feature values and the trained model generated by the generation unit 12 (Step S204). Note that the information processing apparatus 10B infers correct answer labels (objective variables) by using the trained model while using the calculated feature values as explanatory variables. In this way, it is possible to, for example, determine the degree of the progress of the condition of the disease of the patient, compare the condition of the disease before a surgery and that after the surgery, and evaluate the degree of recovery.

Next, the output unit 17 of the information processing apparatus 10B outputs information based on the result of the decision made by the determination unit 16 (Step S205). Note that the information processing apparatus 10B may display, for example, a message indicating the presence/absence of an abnormality and the severity thereof. Further, the information processing apparatus 10B may report the presence/absence of an abnormality and the severity thereof by, for example, producing a sound (or a voice) and/or turning on a lamp or like. Further, the information processing apparatus 10B may record, for example, changes in the value indicating the degree of certainty of the presence/absence of an abnormality for a specific user, calculated in the step S204 at a plurality of dates/times (e.g., on a weekly basis). Then, the information processing apparatus 10B may output a message about advice on rehabilitation for the user based on the changes.

(Effect of Each Item Used for Inference)

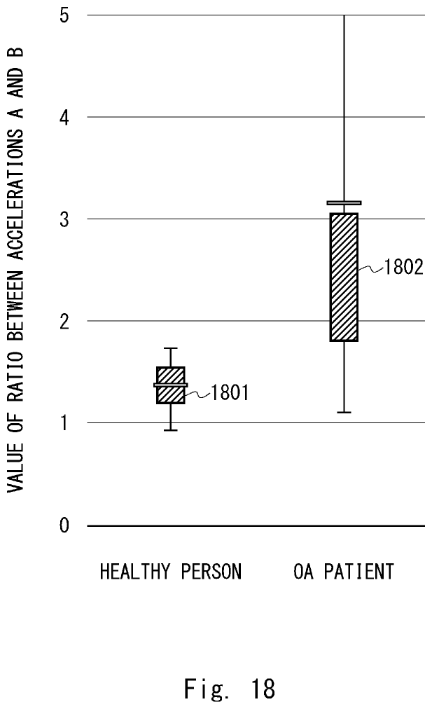

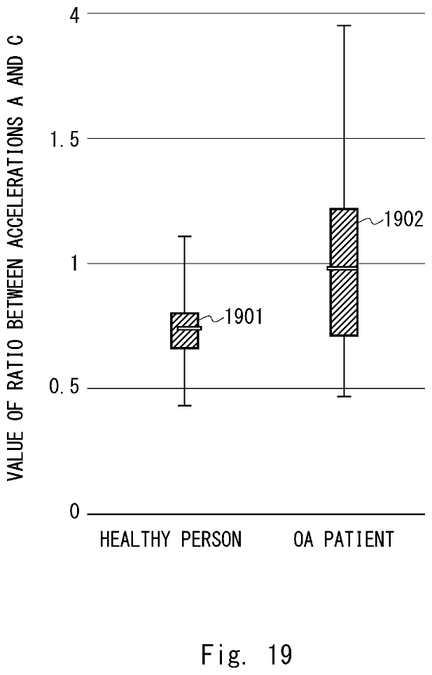

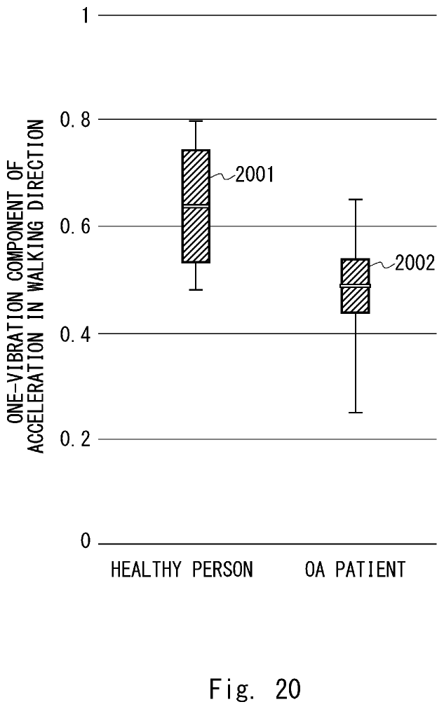

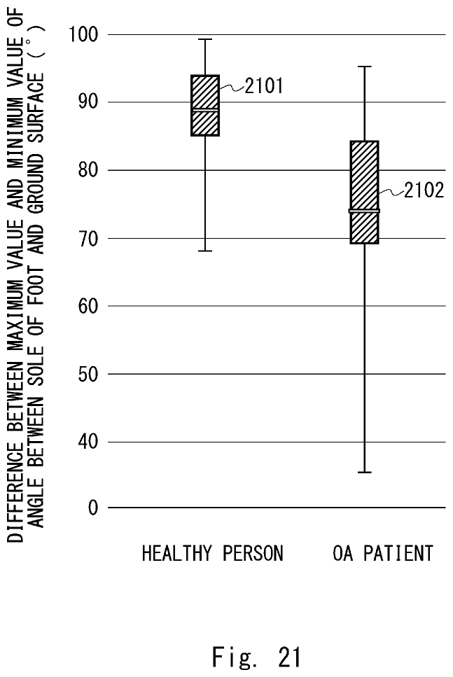

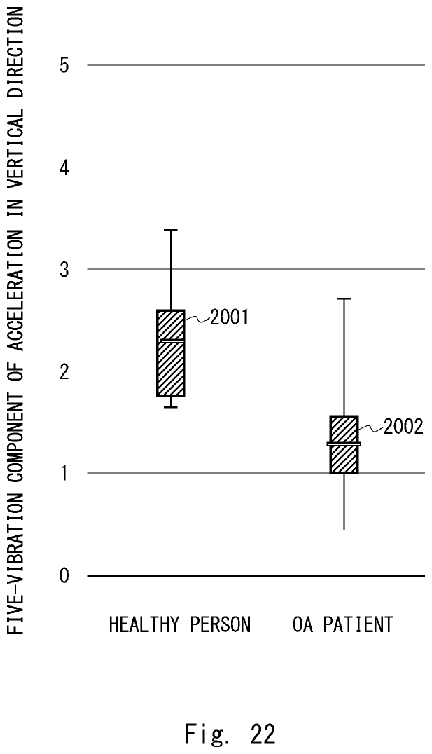

Next, an example of the effect of each item (each feature value or each explanatory variable) used for inference will be described with reference to FIGS. 18 to 22. FIG. 18 shows an example of the value of a ratio between accelerations A and B during a period in which a healthy person makes one step and that during a period in which a patient makes one step based on data measured by the sensor 21 according to the example embodiment. FIG. 19 shows an example of the value of a ratio between accelerations A and C during a period in which a healthy person makes one step and that during a period in which a patient makes one step based on data measured by the sensor 21 according to the example embodiment. FIG. 20 shows an example of a one-vibration component of an acceleration in the walking direction during a period in which a healthy person makes one step and that during a period in which a patient makes one step based on data measured by the sensor 21 according to the example embodiment. FIG. 21 shows an example of a difference between the maximum value and the minimum value of an angle between the sole of a foot and the ground surface in the walking direction during a period in which a healthy person makes one step and that during a period in which a patient makes one step based on data measured by the sensor 21 according to the example embodiment. FIG. 22 shows an example of a five-vibration component of an acceleration in the vertically upward direction during a period in which a healthy person makes one step and that during a period in which a patient makes one step based on data measured by the sensor 21 according to the example embodiment.

FIGS. 18 to 22 show examples of measurement data for each item, obtained from 9 elderly healthy persons and 16 OA patients. Note that the value for each item for the group of healthy persons and that for the group of OA patients are extracted from waveforms each of which has been normalized for each step. Further, for each of the groups, an average and a variance of all the steps for all the users in the group are calculated. The comparison for the value for each item has been performed through a two sided test using a t-distribution. Note that, in the following description, the P-value indicates a probability that the test statistic will have that value under the null hypothesis in a statistical hypothesis test. The smaller the P-value is, the less likely it indicates that the test statistic will have that value. Further, it is considered that when the P-value is lower than 5% (P<0.05. significance level of 5%), there is a difference between the two groups.

FIG. 18 shows an example of a value 1801 of a ratio between accelerations A and B in the group of healthy persons, and a value 1802 of a ratio between accelerations A and B in the group of OA patients. The P-value in the case shown in FIG. 18 is about 0.004. FIG. 19 shows an example of a value 1901 of a ratio between accelerations A and C in the group of healthy persons, and a value 1902 of a ratio between accelerations A and C in the group of OA patients. The P-value in the case shown in FIG. 19 is about 0.002.

FIG. 20 shows an example of a one-time vibration component 2001 of an acceleration in the walking direction during a period in which a person belonging to the healthy-person group makes one step, and a one-time vibration component 2002 of an acceleration in the walking direction during a period in which a person (i.e., a patient) belonging to the OA-patient group makes one step. The P-value in the case shown in FIG. 20 is lower than 0.001. FIG. 21 shows an example of a difference 2101 between the maximum value and the minimum value of the angle between the sole of a foot and the ground surface in the walking direction during a period in which a person belonging to the healthy-person group makes one step, and a difference 2102 between the maximum value and the minimum value of the angle between the sole of a foot and the ground surface in the walking direction during a period in which a person belonging to the OA-patient group makes one step. The P-value in the case shown in FIG. 21 is lower than 0.001.

FIG. 22 shows an example of a five-time vibration component 2201 of an acceleration of a user in the vertical direction during a period in which a person (i.e., the user) belonging to the healthy-person group makes one step, and a five-time vibration component 2202 of an acceleration of a user in the vertical direction during a period in which a person (i.e., the user) belonging to the OA-patient group makes one step. The P-value in the case shown in FIG. 22 is lower than 0.001.

### <Modified Example>

The information processing apparatus 10 may be an apparatus housed in one housing, but the information processing apparatus 10 according to the present disclosure is not limited to such examples. Each unit of the information processing apparatus 10 may be implemented by, for example, cloud computing formed by at least one computer. Further, each unit of the information processing apparatus 10 may be implemented by two of more of the measurement apparatuses 20, the user terminal 30, the server 40, and hospital terminal 50. Such an information processing apparatus 10 is also included in examples of the “information processing apparatus” according to the present disclosure.

Each of above-described example embodiments can be combined as desirable by one of ordinary skill in art.

An example advantage according to above-described example embodiments is to be able to make a proper decision about an abnormality in a lower limb.

While the disclosure has been particularly shown and described with reference to example embodiments thereof, the disclosure is not limited to these example embodiments. It will be understood by those of ordinary skill in art that various changes in form and details may be made therein without departing from the spirit and scope of the present disclosure as defined by the claims.

The whole or part of the example embodiments disclosed above can be described as, but not limited to, the following supplementary notes.

### (Supplementary Note 1)

An information processing apparatus comprising:
an acquisition unit configured to acquire information based on a sensor attached to a foot of a user;
a determination unit configured to make a decision about an abnormality in walking of the user based on an acceleration of the user in a walking direction, acquired by the acquisition unit; and
an output unit configured to output information based on a result of the decision made by the determination unit.

### (Supplementary Note 2)

The information processing apparatus described in Supplementary note 1, wherein the acquisition unit acquires information based on a sensor attached at a position located between an arch of the user's foot and a heel thereof.

### (Supplementary Note 3)

The information processing apparatus described in Supplementary note 1 or 2, wherein the determination unit makes a decision about an abnormality in walking of the user based on at least one of a first acceleration in the walking direction that occurs when a toe of the foot comes off a ground surface, a second acceleration in the walking direction that occurs when the foot is swung in the walking direction by contraction of a thigh muscle of the user, and a walking speed of the user.

### (Supplementary Note 4)

The information processing apparatus described in any one of

Supplementary notes 1 to 3, wherein the determination unit makes a decision about an abnormality in walking of the user based on at least one of a third acceleration in the walking direction that occurs when the foot lands on a ground surface, a second acceleration in the walking direction that occurs when the foot is swung in the walking direction by contraction of a thigh muscle of the user, and a walking speed of the user.

### (Supplementary Note 5)

The information processing apparatus described in any one of Supplementary notes 1 to 4, wherein the determination unit makes a decision about an abnormality in walking of the user based on a magnitude of a one-vibration component of an acceleration of the user in the walking direction during a period in which the user makes one step.

### (Supplementary Note 6)

The information processing apparatus described in any one of Supplementary notes 1 to 5, wherein the determination unit makes a decision about an abnormality in walking of the user based on at least one of an angle between a sole of the foot and a ground surface in the walking direction during a period in which the user makes one step, and a magnitude of a five-vibration component of an acceleration of the user in a vertical direction during the period in which the user makes one step.

### (Supplementary Note 7)

An information processing method comprising:
acquiring information based on a sensor attached to a foot of a user;
making a decision about an abnormality in walking of the user based on an acquired acceleration of the user in a walking direction; and
outputting information based on a result of the decision.

### (Supplementary Note 8)

A program for causing an information processing apparatus to perform:
a process for acquiring information based on a sensor attached to a foot of a user;
a process for making a decision about an abnormality in walking of the user based on an acquired acceleration of the user in a walking direction; and
a process for outputting information based on a result of the decision.

### (Supplementary Note 9)

A trained model configured to cause an information processing apparatus to perform a process for making a decision about an abnormality in walking of a user based on an acceleration of the user in a walking direction, measured by a sensor attached to a foot of the user.

### (Supplementary Note 10)

An information processing system comprising a sensor attached to a foot of a user, an information processing apparatus, and an information processing terminal, wherein
the information processing system comprises:
an acquisition unit configured to acquire information based on an acceleration of the user in a walking direction, measured by the sensor;
a determination unit configured to make a decision about an abnormality in walking of the user based on an acceleration of the user in a walking direction, acquired by the acquisition unit; and
an output unit configured to make the information processing terminal output information based on a result of the decision made by the determination unit.

### (Supplementary Note 11)

The information processing system described in Supplementary note 10, wherein the acquisition unit acquires information based on a sensor attached at a position located between an arch of the user's foot and a heel thereof.

### (Supplementary Note 12)

A generation method comprising:
acquiring a data set containing a combination of information based on an acceleration of a user in a walking direction, measured by a sensor attached to a foot of the user, and information indicating an abnormality in walking of the user;
generating, based on acquired information, a trained model from information measured by the sensor attached to the foot of the user, the trained model being configured to make a decision about an abnormality in walking of the user; and
outputting the generated trained model.

### (Supplementary Note 13)

An information processing apparatus comprising:
an acquisition unit configured to acquire a data set containing a combination of information based on an acceleration of a user in a walking direction, measured by a sensor attached to a foot of the user, and information indicating an abnormality in walking of the user;
a generation unit configured to generate, based on the acceleration of the user in the walking direction, acquired by the acquisition unit, a trained model from information measured by the sensor attached to the foot of the user, the trained model being configured to make a decision about an abnormality in walking of the user; and
an output unit configured to output the trained model generated by the generation unit.

### (Supplementary Note 14)

The information processing apparatus described in Supplementary note 13, wherein the acquisition unit acquires a data set containing a combination of information based on the acceleration of the user in the walking direction, measured by a sensor attached at a position located between an arch of the user's foot and a heel thereof, and information indicating an abnormality in walking of the user.

### (Supplementary Note 15)

A program for causing an information processing apparatus to perform:
a process for acquiring a data set containing a combination of information based on an acceleration of a user in a walking direction, measured by a sensor attached to a foot of the user, and information indicating an abnormality in walking of the user;
a process for generating, based on acquired information, a trained model from information measured by the sensor attached to the foot of the user, the trained model being configured to make a decision about an abnormality in walking of the user; and
a process for outputting the generated trained model.

### Reference Signs List

1 INFORMATION PROCESSING SYSTEM

10 INFORMATION PROCESSING APPARATUS

11 ACQUISITION UNIT

12 GENERATION UNIT

13 OUTPUT UNIT

15 ACQUISITION UNIT

16 DETERMINATION UNIT

17 OUTPUT UNIT

20 MEASUREMENT APPARATUS

21 SENSOR

22 CONTROL UNIT

23 COMMUNICATION UNIT

30 USER TERMINAL

40 SERVER

50 HOSPITAL TERMINAL

What is claimed is:

1. An information processing apparatus comprising:
an acquisition unit configured to acquire information based on a sensor attached to a foot of a user;
a determination unit configured to make a decision about an abnormality in walking of the user based on an acceleration of the user in a walking direction, acquired by the acquisition unit; and
an output unit configured to output information based on a result of the decision made by the determination unit.

2. The information processing apparatus according to claim 1, wherein the acquisition unit acquires information based on a sensor attached at a position located between an arch of the user's foot and a heel thereof.

3. The information processing apparatus according to claim 1, wherein the determination unit makes a decision about an abnormality in walking of the user based on at least one of a first acceleration in the walking direction that occurs when a toe of the foot comes off a ground surface, a second acceleration in the walking direction that occurs when the foot is swung in the walking direction by contraction of a thigh muscle of the user, and a walking speed of the user.

4. The information processing apparatus according to claim 1, wherein the determination unit makes a decision about an abnormality in walking of the user based on at least one of a third acceleration in the walking direction that occurs when the foot lands on a ground surface, a second acceleration in the walking direction that occurs when the foot is swung in the walking direction by contraction of a thigh muscle of the user, and a walking speed of the user.

5. The information processing apparatus according to claim 1, wherein the determination unit makes a decision about an abnormality in walking of the user based on a magnitude of a one-vibration component of an acceleration of the user in the walking direction during a period in which the user makes one step.

6. The information processing apparatus according to claim 1, wherein the determination unit makes a decision about an abnormality in walking of the user based on at least one of an angle between a sole of the foot and a ground surface in the walking direction during a period in which the user makes one step, and a magnitude of a five-vibration component of an acceleration of the user in a vertical direction during the period in which the user makes one step.

7. An information processing method comprising:
acquiring information based on a sensor attached to a foot of a user;
making a decision about an abnormality in walking of the user based on an acquired acceleration of the user in a walking direction; and
outputting information based on a result of the decision.

8. An information processing system comprising a sensor attached to a foot of a user, an information processing apparatus, and an information processing terminal, wherein
the information processing system comprises:
an acquisition unit configured to acquire information based on an acceleration of the user in a walking direction, measured by the sensor;
a determination unit configured to make a decision about an abnormality in walking of the user based on an acceleration of the user in a walking direction, acquired by the acquisition unit; and
an output unit configured to make the information processing terminal output information based on a result of the decision made by the determination unit.
